(19) United States
(12) Patent Application Publication
(10) Pub. No.: US 2023/0098120 A1
(43) Pub. Date: Mar. 30, 2023

(54) PAYMENT INFORMATION MANAGEMENT APPARATUS, CHECKOUT SYSTEM, AND PAYMENT INFORMATION MANAGEMENT METHOD

(71) Applicant: NEC Corporation, Minato-ku, Tokyo (JP)

(72) Inventors: Yukio NADA, Tokyo (JP); Sayuri WATANABE, Tokyo (JP); Toshinobu OGATSU, Tokyo (JP); Masamichi TANABE, Tokyo (JP)

(73) Assignee: NEC Corporation, Minato-ku, Tokyo (JP)

(21) Appl. No.: 17/802,689

(22) PCT Filed: Mar. 1, 2021
(86) PCT No.: PCT/JP2021/007640

§ 371 Date: Aug. 26, 2022

(30) Foreign Application Priority Data
Mar. 2, 2020 (JP) 2020-034919

(51) Int. Cl. G06Q 20/40

(52) U.S. Cl. CPC G06Q 20/40145

(57) ABSTRACT

A first registration unit stores payment information and master face information in a first storage unit in association with each other. The payment information is information necessary for electronically making payment. An acquisition unit acquires, from a checkout apparatus, face information of a customer. An authentication unit reads, when master face information in which a score for the face information is equal to or more than a reference value is present, payment information relevant to the master face information from the first storage unit, and causes the checkout apparatus to execute checkout processing using the payment information. A second registration unit stores, in the first storage unit in association with payment information of a customer, as master biometric information, biometric information including iris information and/or vein information of the customer, under a necessary condition in that the checkout apparatus executes the checkout processing using the payment information.

### TECHNICAL FIELD

The present invention relates to a payment information management apparatus, a checkout system, a payment information management method, and a program.

### BACKGROUND ART

In recent years, in electronic payment, personal authentication using biometric information is performed. Patent Document 1, for example, describes that an ID and biometric information of a user are stored in association with each other and authentication of a user is performed by using the biometric information. In Patent Document 1, payment is made after authentication of a user is successful.

Further, Patent Document 2 describes that face authentication and iris authentication are performed in parallel, and when either of the face authentication and the iris authentication is successful, it is determined that personal authentication is successful.

### RELATED DOCUMENT

### Patent Document

[Patent Document 1] Japanese Patent Application Publication No. 2019-67075

[Patent Document 2] Japanese Patent Application Publication No. 2005-242677

### SUMMARY OF THE INVENTION

### Technical Problem

As one of biometric authentication, face authentication is available. Face authentication has an advantage in which a load on an authentication target person is small, including registration of master information. However, when very-high authentication accuracy is required, face authentication may not always satisfy this demand. As another biometric authentication, iris authentication and vein authentication are available. Authentication using these pieces of biometric information has an advantage in which authentication accuracy is high. On the other hand, in iris authentication and vein authentication, a dedicated image-capture unit is required, and therefore it is necessary for an authentication target person to go to a location where there is a dedicated image-capture unit when master information is registered.

One of objects of the present invention is to enable, while an advantage of convenience of face authentication is taken, master information to be easily registered in biometric authentication using iris authentication or vein authentication. Further, another object of the present invention is to achieve authentication accuracy and convenience with each other by using together face authentication, and iris authentication or vein authentication.

### Solution to Problem

According to the present invention, provided is a payment information management apparatus including:

a first registration unit that acquires, from a first terminal, payment information being information necessary for electronically making payment and master face information indicating a face of a person relevant to the payment information, and stores the payment information and the master face information in a first storage unit in association with each other;

an acquisition unit that acquires, from a checkout apparatus installed in a store, authentication-use face information being face information of a customer about to make a checkout by using the checkout apparatus;

an authentication unit that reads, when the master face information in which a score for the authentication-use face information is equal to or more than a reference value is present, the payment information relevant to the master face information, and causes the checkout apparatus to execute checkout processing using the payment information; and

a second registration unit that stores, in the first storage unit in association with the payment information of the customer, as master biometric information, biometric information including iris information and/or vein information of the customer generated by the checkout apparatus, under a necessary condition in that the checkout apparatus executes the checkout processing using the payment information.

According to the present invention, provided is a payment information management apparatus

being used together with a first storage unit that stores payment information being information necessary for electronically making payment, master face information indicating a face of a person relevant to the payment information, and master biometric information indicating iris information and/or vein information of the person in association with one another, the apparatus including:

an acquisition unit that acquires, from a checkout apparatus installed in a store, authentication-use face information being face information of a customer about to make a checkout by using the checkout apparatus and authentication-use biometric information being the biometric information of the customer; and

an authentication unit that performs authentication processing by using the first storage unit, and the authentication-use biometric information and/or the authentication-use face information, wherein

the authentication unit
reads, when the master face information in which a score for the authentication-use face information is equal to or more than a reference value is present, the payment information relevant to the master face information, and causes the checkout apparatus to execute checkout processing using the payment information, and
determines, when the master face information in which a score for the authentication-use face information is equal to or more than a reference value is absent, the master biometric information in which a score for the authentication-use biometric information is equal to or more than a reference value, reads the payment information relevant to the master biometric information, and causes the checkout apparatus to execute checkout processing using the payment information.

According to the present invention, provided is a checkout system including:

a checkout apparatus used in a store; and

a payment information management apparatus that manages information used in the checkout apparatus, wherein

the payment information management apparatus includes
a first registration unit that acquires, from a first terminal, payment information being information necessary for electronically making payment and master face information indicating a face of a person relevant to the payment information, and stores the payment information and the master face information in a first storage unit in association with each other,

the checkout apparatus includes
a face information generation unit that generates authentication-use face information being face information of a customer about to make a checkout by using the checkout apparatus,
a biometric information generation unit that generates biometric information including iris information and/or vein information of the customer, and
a transmission unit that transmits, to the payment information management apparatus, the authentication-use face information and the biometric information, and

the payment information management apparatus further includes
an acquisition unit that acquires, from the checkout apparatus, the authentication-use face information and the biometric information,
an authentication unit that reads, when the master face information in which a score for the authentication-use face information is equal to or more than a reference value is present, the payment information relevant to the master face information, and causes the checkout apparatus to execute checkout processing using the payment information, and
a second registration unit that stores, in the first storage unit in association with the payment information of the customer, as master biometric information, the biometric information acquired from the checkout apparatus, under a necessary condition in that the checkout apparatus executes the checkout processing using the payment information.

According to the present invention, provided is a payment information management method including,

by a computer:
acquiring, from a first terminal, payment information being information necessary for electronically making payment and master face information indicating a face of a person relevant to the payment information, and storing the payment information and the master face information in a first storage unit in association with each other;
acquiring, from a checkout apparatus installed in a store, authentication-use face information being face information of a customer about to make a checkout by using the checkout apparatus;
reading, when the master face information in which a score for the authentication-use face information is equal to or more than a reference value is present, the payment information relevant to the master face information, and causing the checkout apparatus to execute checkout processing using the payment information; and
storing, in the first storage unit in association with the payment information of the customer, as master biometric information, biometric information being information generated by the checkout apparatus and including iris information and/or vein information of the customer, under a necessary condition in that the checkout apparatus executes the checkout processing using the payment information.

According to the present invention, provided is a program causing a computer to include:

a function of acquiring, from a first terminal, payment information being information necessary for electronically making payment and master face information indicating a face of a person relevant to the payment information, and storing the payment information and the master face information in a first storage unit in association with each other;

a function of acquiring, from a checkout apparatus installed in a store, authentication-use face information being face information of a customer about to make a checkout by using the checkout apparatus;

a function of reading, when the master face information in which a score for the authentication-use face information is equal to or more than a reference value is present, the payment information relevant to the master face information, and causing the checkout apparatus to execute checkout processing using the payment information; and

a function of storing, in the first storage unit in association with the payment information of the customer, as master biometric information, biometric information being information generated by the checkout apparatus and including iris information and/or vein information of the customer, under a necessary condition in that the checkout apparatus executes the checkout processing using the payment information.

### Advantageous Effects of Invention

According to a certain invention, while an advantage of convenience of face authentication is taken, in iris authentication and/or vein authentication, master biometric information is easily registered. Further, according to another invention, face authentication, and iris authentication and/or vein information are used together, and thereby, authentication accuracy and convenience can be achieved with each other.

### BRIEF DESCRIPTION OF THE DRAWINGS

FIG. 1 is a diagram illustrating a configuration of a checkout system according to a first example embodiment.

FIG. 2 is a diagram illustrating one example of a function configuration of a payment information management apparatus.

FIG. 3 is a diagram illustrating one example of a function configuration of a checkout apparatus.

FIG. 4 is a diagram illustrating one example of a function configuration of a first terminal.

FIG. 5 is a diagram illustrating one example of a data configuration of a first storage unit of the payment information management apparatus.

FIG. 6 is a diagram illustrating a hardware configuration example of the payment information management apparatus.

FIG. 7 is a flowchart illustrating first processing performed by the payment information management apparatus.

FIG. 8 is a flowchart illustrating second processing performed by the payment information management apparatus.

FIG. 9 is a flowchart illustrating third processing performed by the payment information management apparatus.

FIG. 10 is a flowchart illustrating fourth processing performed by the payment information management apparatus.

FIG. 11 is a diagram illustrating a usage environment of a payment information management apparatus according to a second example embodiment.

### DESCRIPTION OF EMBODIMENTS

Hereinafter, example embodiments according to the present invention are described by using the accompanying drawings. Note that, in all drawings, a similar component is assigned with a similar reference sign, and description thereof is not repeated, as appropriate.

### First Example Embodiment

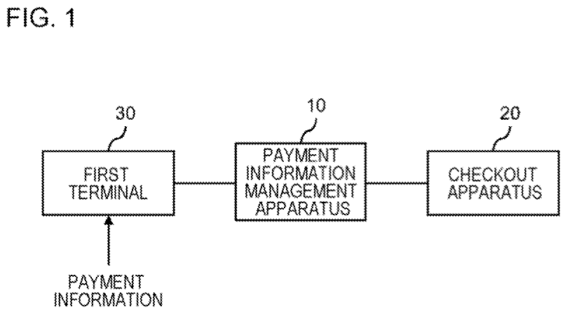

FIG. 1 is a diagram illustrating a configuration of a checkout system according to the present example embodiment. The checkout system according to the example embodiment includes a payment information management apparatus 10 and a checkout apparatus 20, and is used together with a first terminal 30.

The checkout apparatus 20 is installed in a store, and is used when a customer makes a checkout of a consideration for a product or a service. At this occasion, the checkout apparatus 20 makes a checkout by using an electronic method, for example, a credit card or an electronic money card. The payment information management apparatus 10 transmits, to the checkout apparatus 20, information (hereinafter, referred to as payment information) necessary for electronically making payment. The payment information is information for determining a payment means and includes at least one of, for example, a number of a credit card and an expiration date of the card, a number of an electronic money card, and an account number of a bank. The payment information management apparatus 10 uses biometric authentication when transmitting payment information to the checkout apparatus 20. The biometric authentication is face authentication, and iris authentication and/or vein authentication. In these pieces of authentication, the payment information management apparatus 10 uses master-use face information (hereinafter, referred to as a master face image), and master-use iris information and/or master-use vein information (hereinafter, referred to as master biometric information).

According to the present example embodiment, face information is, for example, a feature value of a face or a face image, iris information is, for example, a feature value of an iris or an iris image, and vein information is, for example, a feature value of a vein or an image of a vein. When a face image is used as face information, face information is processed into a feature value of a face, as necessary. Further, when an iris image is used as iris information, iris information is processed into a feature value of an iris, as necessary. Furthermore, when an image of a vein is used as vein information, vein information is processed into a feature value of a vein, as necessary. Note that, in the following description, with regard to “iris authentication and/or vein authentication”, iris authentication is exemplified. Similarly, with regard to “iris information and/or vein information”, iris information is exemplified. Further similarly, with regard to “an iris image and/or an image of a vein”, an iris image is exemplified.

A person using the checkout system previously registers payment information and master face information in the payment information management apparatus 10. For the registration, a first terminal 30 is used. The first terminal 30 may be a mobile terminal, or may be a terminal installed in a store or the like, for example, a kiosk terminal. And, the first terminal 30 includes an image-capture unit (a part or a whole of a face information generation unit 310 to be described later) for photographing a face. The master face information is an image generated by the image-capture unit or a feature value extracted from the image.

Further, the checkout apparatus 20 includes an image-capture unit (a part or a whole of a face information generation unit 210 to be described later) for capturing a face image of a customer and an image-capture unit (a part or a whole of a biometric information generation unit 220 to be described later) for capturing an iris image of the customer. Images generated by these image-capture units are used when personal authentication is performed in checkout processing. Further, an iris image generated by the checkout apparatus 20 is also used when master biometric information is generated.

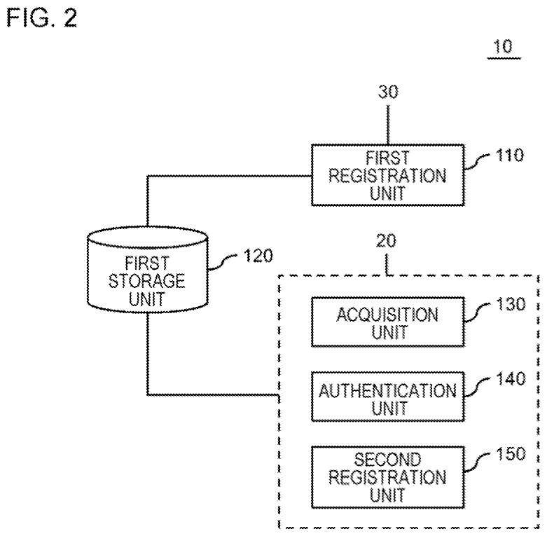

FIG. 2 is a diagram illustrating one example of a function configuration of the payment information management apparatus 10. The payment information management apparatus 10 includes a first registration unit 110, an acquisition unit 130, an authentication unit 140, and a second registration unit 150.

The first registration unit 110 acquires, from the first terminal 30, payment information and master face information as described above, and stores, in a first storage unit 120, the payment information and the master face information in association with each other. The acquisition unit 130 acquires, from the checkout apparatus 20, face information (hereinafter, referred to as authentication-use face information) of a customer about to make a checkout by using the checkout apparatus 20. The authentication unit 140 reads, when master face information in which a score for authentication-use face information is equal to or more than a reference value is present, payment information relevant to the master face information from the first storage unit 120, and causes the checkout apparatus 20 to execute checkout processing using the payment information. The second registration unit 150 stores, in the first storage unit 120 in association with payment information of a customer, as master biometric information, iris information of the customer, under a necessary condition in that the checkout apparatus 20 executes the checkout processing using the payment information. The master biometric information is generated by the checkout apparatus 20. Note that, details of processing performed by each function unit of the payment information management apparatus 10 are described later by using a flowchart.

In the example illustrated in the present figure, the first storage unit 120 is a part of the payment information management apparatus 10. However, the first storage unit 120 may be located outside the payment information management apparatus 10.

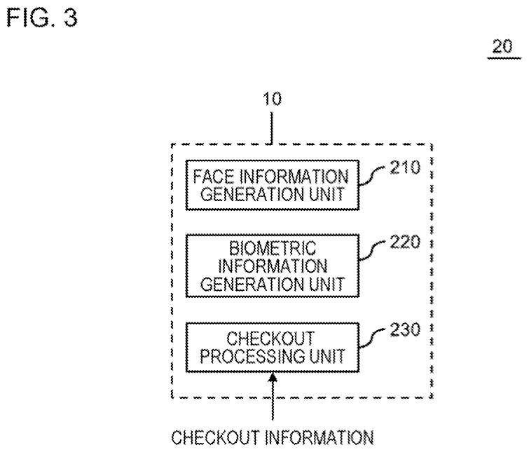

FIG. 3 is a diagram illustrating one example of a function configuration of the checkout apparatus 20. The checkout apparatus 20 includes the face information generation unit 210, the biometric information generation unit 220, and a checkout processing unit 230.

The face information generation unit 210 includes an image-capture unit, for example, a visible-light image-capture unit for generating a face image of a customer. The face information generation unit 210 transmits, to the payment information management apparatus 10, the face image or a feature value generated from the face image as information for generating face information or as face information itself.

The biometric information generation unit 220 includes an image-capture unit, for example, a near-infrared image-capture unit for generating an iris image of a customer. The biometric information generation unit 220 transmits, to the payment information management apparatus 10, the iris image or a feature value generated from the iris image as information for generating iris information or as iris information itself.

The checkout processing unit 230 acquires information (hereinafter, referred to as checkout information) including a consideration to be paid by a customer, and also acquires payment information of the customer from the payment information management apparatus 10. Then, the checkout processing unit 230 performs processing for generating, by a checkout means indicated by the acquired payment information, payment for an amount of money indicated by the acquired checkout information.

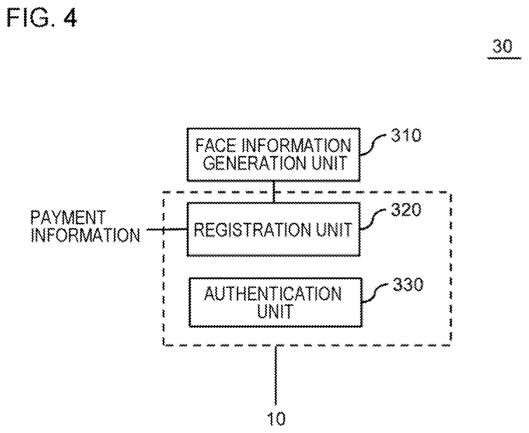

FIG. 4 is a diagram illustrating one example of a function configuration of the first terminal 30. The first terminal 30 includes the face information generation unit 310 and a registration unit 320.

The face information generation unit 310 includes an image-capture unit, for example, a visible-light image-capture unit for generating a face image of a person. The registration unit 320 acquires payment information of a person, and also transmits, to the payment information management apparatus 10, the payment information in association with a face image (or a feature value generated from the face image) generated by the face information generation unit 310. The face image or the feature value of the face transmitted from the first terminal 30 is used as information for generating master face information or as master face information.

Further, the registration unit 320 acquires attribute information of the person, for example, at least one of age or an age-group, gender, a name, and an address, and transmits the acquired information to the face information generation unit 310 in association with a face image.

In the example illustrated in the present figure, the first terminal 30 further includes an approval unit 330. The approval unit 330 causes a person relevant to iris information acquired by the payment information management apparatus 10 to input information indicating whether to be able to use the iris information for authentication processing as a master iris image. The approval unit 330 transmits an input result to the payment information management apparatus 10. Then, the payment information management apparatus 10 causes, when acquiring information that authentication processing can be performed from the first terminal 30, authentication processing using the iris information to be executable.

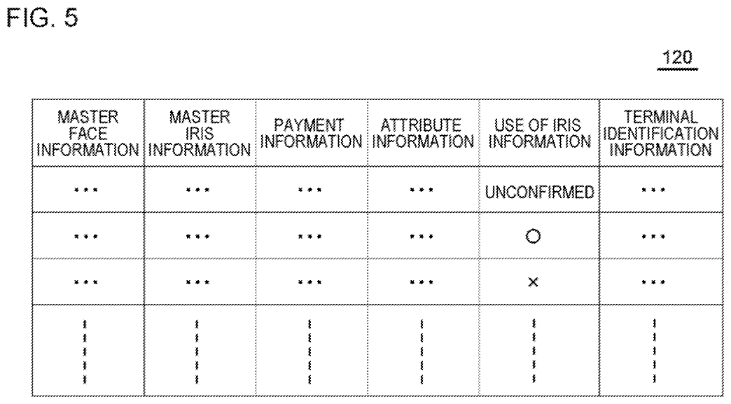

FIG. 5 is a diagram illustrating one example of a data configuration of the first storage unit 120 of the payment information management apparatus 10. In the present figure, the first storage unit 120 stores master face information, master biometric information, payment information, attribute information, and information indicating whether authentication using iris information can be performed in association with one another. Herein, when whether authentication using iris information can be performed is not yet confirmed, the first storage unit 120 stores information indicating being unconfirmed as information indicating whether authentication using iris information can be performed.

Further, the first storage unit 120 further stores terminal identification information in association with face information. The terminal identification information is information for communicating with a terminal carried by a person relevant to face information, and is, for example, a mail address, an account of SNS, or the like. The terminal identification information is acquired, for example, via input to the first terminal 30 by the person. When the first terminal 30 is a mobile terminal of the person, a terminal indicated by terminal identification information may be the first terminal 30.

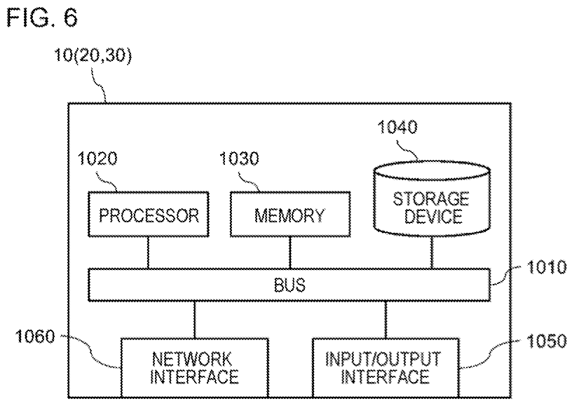

FIG. 6 is a diagram illustrating a hardware configuration example of the payment information management apparatus 10. The payment information management apparatus 10 includes a bus 1010, a processor 1020, a memory 1030, a storage device 1040, an input/output interface 1050, and a network interface 1060.

The bus 1010 is a data transmission path through which the processor 1020, the memory 1030, the storage device 1040, the input/output interface 1050, and the network interface 1060 transmit/receive data to/from one another. However, a method of mutually connecting the processor 1020 and the like is not limited to bus connection.

The processor 1020 is a processor achieved by a central processing unit (CPU), a graphics processing unit (GPU), or the like.

The memory 1030 is a main storage achieved by a random access memory (RAM) or the like.

The storage device 1040 is an auxiliary storage achieved by a hard disk drive (HDD), a solid state drive (SSD), a memory card, a read only memory (ROM), or the like. The storage device 1040 stores a program module for achieving each function (e.g., the first registration unit 110, the acquisition unit 130, the authentication unit 140, and the second registration unit 150) of the payment information management apparatus 10. The processor 1020 reads each of the program modules onto the memory 1030 and executes the read program module, and thereby achieves each function relevant to the program module. Further, the storage device 1040 also functions as the first storage unit 120.

The input/output interface 1050 is an interface for connecting the payment information management apparatus 10 and various types of input/output devices.

The network interface 1060 is an interface for connecting the payment information management apparatus 10 to a network. The network is, for example, a local area network (LAN) or a wide area network (WAN). A method for connecting to the network based on the network interface 1060 may be wireless connection or may be wired connection. The payment information management apparatus 10 may communicate with the checkout apparatus 20 and the first terminal 30 via the network interface 1060.

Note that, a hardware configuration of each of the checkout apparatus 20 and the first terminal 30 is also similar to the example illustrated in FIG. 6.

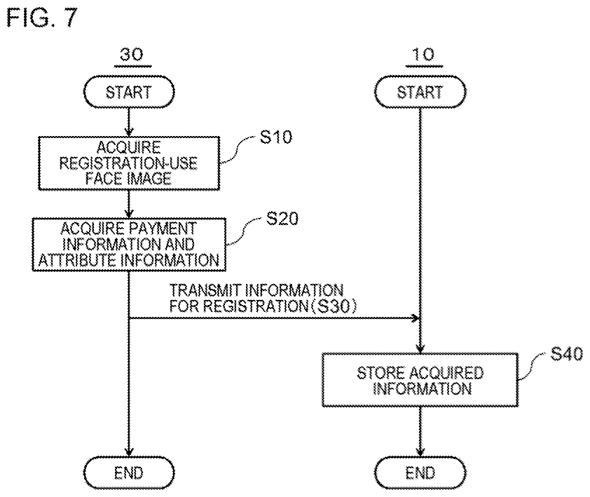

FIG. 7 is a flowchart illustrating first processing performed by the payment information management apparatus 10. The processing illustrated in the present figure is processing for registering, in the first storage unit 120, master face information, together with payment information, attribute information, and terminal identification information, and is performed together with the first terminal 30.

First, a person using the first terminal 30 photographs a face image by using the face information generation unit 310 (step S10). Further, the person inputs, to the registration unit 320, payment information, attribute information, and terminal identification information (step S20). Then, the registration unit 320 transmits, to the payment information management apparatus 10, the face image or a feature value generated by the face information generation unit 310 in association with the payment information, the attribute information, and the terminal identification information (step S30).

The first registration unit 110 of the payment information management apparatus 10 stores, in the first storage unit 120, the face image or the feature value acquired from the first terminal 30 as face information. Herein, the first registration unit 110 may process, when acquiring a face image from the first terminal 30, the face image, generate a feature value of the face, and thereby store the feature value in the first storage unit 120 as face information. At this occasion, the first registration unit 110 stores, in the first storage unit 120, the payment information, the attribute information, and the terminal identification information in association with the face information (step S40). In this stage, the first storage unit 120 has not yet stored master biometric information relevant to the person. Further, a column of “information indicating whether to be able to use iris information for authentication processing” of the first storage unit 120 is vacant, or stores information indicating being unconfirmed.

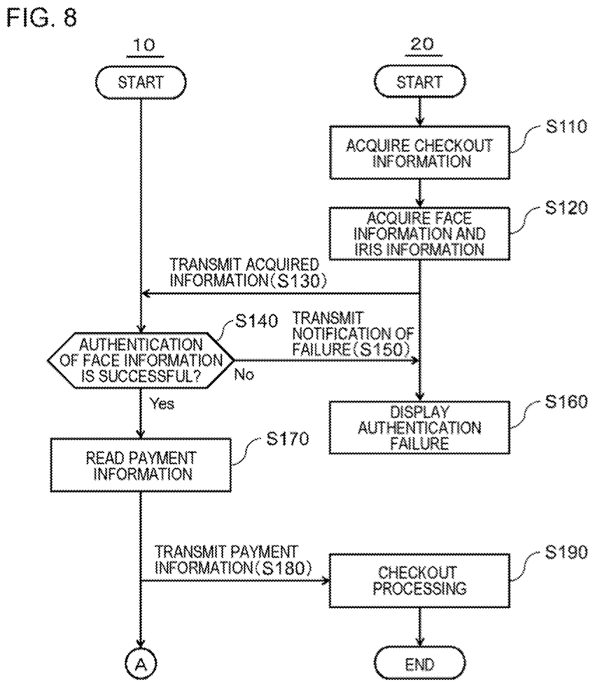

FIG. 8 is a flowchart illustrating second processing performed by the payment information management apparatus 10. The processing illustrated in the present figure indicates a time at which a person having registered, in the first storage unit 120, face information and payment information first makes a checkout as a customer by using the checkout apparatus 20.

First, a customer stands in front of the checkout apparatus 20. Further, the checkout processing unit 230 of the checkout apparatus 20 acquires checkout information of a customer (step S110). When the checkout apparatus 20 includes a product registration function, the checkout apparatus 20 generates checkout information. On the other hand, when the checkout apparatus 20 does not include a product registration function, the checkout processing unit 230 acquires checkout information from an external apparatus (e.g., a product registration apparatus) of the checkout apparatus 20.

Next, the face information generation unit 210 of the checkout apparatus 20 generates a face image of a customer, and generates, by using the face image, authentication-use face information. Further, the biometric information generation unit 220 of the checkout apparatus 20 generates an iris image of a customer, and generates, by using the iris image, iris information (step S120). The iris information can be used as master biometric information. At this occasion, the face information generation unit 210 processes a face image, and thereby, confirms an eye position of a customer. Then, the biometric information generation unit 220 controls a direction of an image-capture unit of the biometric information generation unit 220, by matching the direction with the eye position. Thereby, the biometric information generation unit 220 can easily generate a high-quality iris image.

Note that, in step S120, the biometric information generation unit 220 performs, in parallel with at least a part of acquisition processing for authentication-use face information, at least a part of acquisition processing for authentication-use iris information. The face information generation unit 210, for example, generates a face image, determines an eye position, and generates a feature value of a face as authentication-use face information. Then, until the feature value satisfies a reference, the processing is repeated. The biometric information generation unit 220 controls, every time the face information generation unit 210 determines an eye position, a direction of the image-capture unit by matching the direction with the position, and generates an iris image. Then, the biometric information generation unit 220 generates iris information by using the generated iris image. By doing so, a time required for step S120 is shortened.

Next, the face information generation unit 210, the biometric information generation unit 220, and the checkout processing unit 230 of the payment information management apparatus 10 transmit, to the payment information management apparatus 10, the acquired information, i.e., the checkout information, the authentication-use face information, and the iris information (step S130).

The acquisition unit 130 of the payment information management apparatus 10 acquires, from the checkout apparatus 20, the checkout information, the authentication-use face information, and the iris information. Then, the authentication unit 140 determines whether master face information in which a score for the authentication-use face information is equal to or more than a reference value is present. In a case of presence (step S140: Yes), the authentication unit 140 determines that authentication is successful, and reads, from the first storage unit 120, payment information relevant to the master face image (step S170). Then, the authentication unit 140 transmits the read payment information to the checkout apparatus 20 (step S180).

Then, the checkout processing unit 230 of the checkout apparatus 20 electronically performs checkout processing, by using the payment information acquired from the payment information management apparatus 10 (step S190).

On the other hand, in step S140, in a case where a master face image satisfying a condition is absent (step S140: No), the authentication unit 140 transmits, to the checkout apparatus 20, information indicating that authentication fails (step S150). Then, the checkout apparatus 20 causes, for example, a display unit of the checkout apparatus 20 to perform display indicating that authentication fails (step S160).

Note that, in processing illustrated in step S120 to step S140, the payment information management apparatus 10 may repeat, until authentication is successful, generation of a face image and authentication processing using face information for a predetermined time. In this case, when authentication is unsuccessful even when a predetermined time elapses, processing illustrated in step S150 and step S160 is performed.

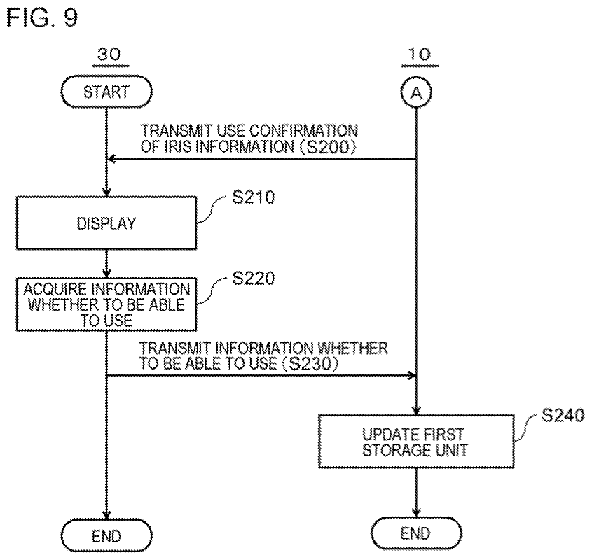

Further, in step S170, the authentication unit 140 confirms, in the first storage unit 120, the column of “information indicating whether to be able to use iris information for authentication processing” relevant to the payment information read in the step S170. When the column is vacant or information indicating being unconfirmed is stored in the column, it is recognized that checkout processing performed this time is first checkout processing in a customer relevant to the processing. In this case, the payment information management apparatus 10 and the first terminal 30 execute processing illustrated in FIG. 9. Note that, the payment information management apparatus 10 and the first terminal 30 may recognize, as a necessary condition for executing processing illustrated in FIG. 9, a fact that master biometric information relevant to the read payment information is not stored in the first storage unit 120

FIG. 9 is a flowchart illustrating third processing performed by the payment information management apparatus 10. The processing illustrated in the present figure is registration processing for a master iris image.

In step S130 in FIG. 8, the biometric information generation unit 220 of the checkout apparatus 20 transmits, to the payment information management apparatus 10, an iris image being able to be a master iris image. The acquisition unit 130 stores the iris image in a column of a master iris image of the first storage unit 20 in association with payment information read in step S170. However, in this stage, the iris image is not handled as a master iris image. In order for the iris image to become a master iris image, it is necessary to store, in FIG. 6, “information indicating that iris information is usable as authentication processing”.

The second registration unit 150 of the payment information management apparatus 10 reads, from the first storage unit 120, terminal identification information relevant to payment information read in step S170 in FIG. 8. Then, the second registration unit 150 transmits, to a terminal of a person, for example, the first terminal 30, by using the terminal identification information, information for confirming whether to be able to perform authentication processing using iris information, i.e., information for confirming whether to be able to store the iris image acquired in step S130 in the first storage unit 120 as a master iris image (step S200).

The first terminal 30 displays a screen for making confirmation by the person (step S210). The person inputs, to the first terminal 30, information indicating whether to be able to use iris information for authentication processing (step S220). Then, the first terminal 30 transmits the information to the payment information management apparatus 10 (step S230).

The second registration unit 150 of the payment information management apparatus 10 updates the first storage unit 120 according to the information acquired from the first terminal 30 (step S240).

The second registration unit 150 stores, when, for example, acquiring information that authentication processing is allowed from the first terminal 30, information for setting iris information as master iris image in the first storage unit 120. Specifically, the second registration unit 150 stores information indicating being usable in the column of “information indicating whether to be able to use iris information for authentication processing” of the first storage unit 120.

On the other hand, the second registration unit 150 stores, when acquiring information that authentication processing is not allowed from the first terminal 30, information for not setting iris information as master iris image in the first storage unit 120. Specifically, the second registration unit 150 stores information indicating being non-usable in the column of “information indicating whether to be able to use iris information for authentication processing” of the first storage unit 120.

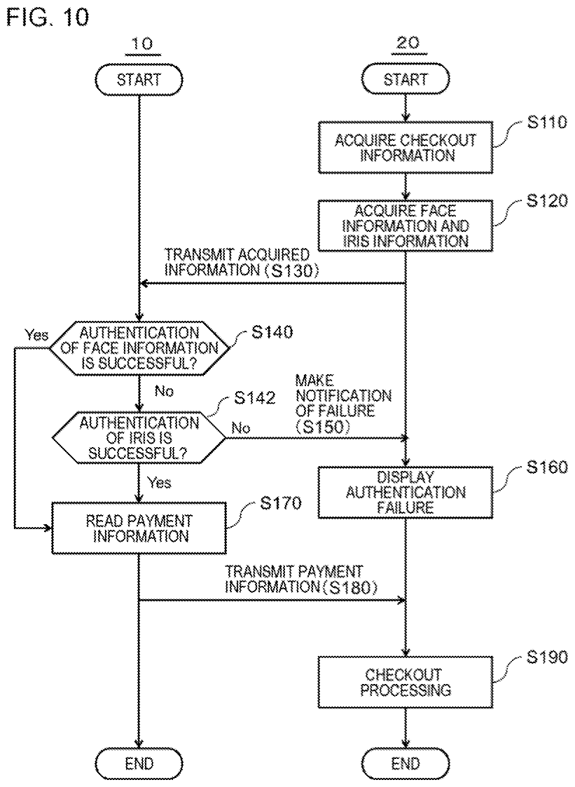

FIG. 10 is a flowchart illustrating fourth processing performed by the payment information management apparatus 10. The processing illustrated in the present figure indicates a time at which a person having registered master biometric information in the first storage unit 120 makes a checkout by using the checkout apparatus 20 as a customer.

Processing illustrated in step S110 to step S140 is similar to FIG. 8. However, iris information transmitted in step S130 is handled as authentication-use iris information.

Then, when authentication using face information is successful (step S140: Yes), processing in and after step S170 is executed, similarly to the example illustrated in FIG. 8.

On the other hand, when authentication using face information fails, i.e., when master face information in which a score for authentication-use face information is equal to or more than a reference value is absent (step S140: No), the authentication unit 140 performs authentication processing using iris information (step S142). For details, the authentication unit 140 determines whether master biometric information in which a score for authentication-use iris information is equal to or more than a reference value is present in the first storage unit 120. In a case of presence (step S142: Yes), the authentication unit 140 determines that authentication is successful, and reads, from the first storage unit 120, payment information relevant to the master iris image (step S170). Note that, in step S142, the authentication unit 140 may select a master face image having a high-order score in step S40, and handle, as a collation target, only a piece of master biometric information relevant to the selected master image.

On the other hand, in step S142, when a master iris image satisfying a condition is absent (step S142: No), processing illustrated in steps S150 and S160 is performed.

Note that, in processing illustrated in step S120, face information may be generated earlier than iris information. In this case, the payment information management apparatus 10 may transmit face information earlier than iris information. Then, the authentication unit 140 of the payment information management apparatus 10 may first start authentication processing using the face information, and thereafter start, at a timing of acquiring the iris information, authentication processing using the iris information. By doing so, a part of authentication processing using iris information can be performed in parallel with authentication processing using face information. Therefore, a time required until step S142 is shortened.

Note that, in the processing illustrated in the present figure, the authentication unit 140 may overwrite, when authentication-use iris information is better in quality than master biometric information (e.g., when being clearer or when a front degree is higher), the master biometric information with the authentication-use iris information used this time.

As described above, according to the present example embodiment, payment information is registered by using the first terminal 30. Herein, information registered by using the first terminal 30 includes master face information but does not include master biometric information. Thus, as the first terminal 30, various terminals such as a mobile terminal are usable. Therefore, a degree of difficulty in registration processing for payment information is lowered.

On the other hand, when payment is made by using the checkout apparatus 20, iris information is generated, and the iris information is registered as master biometric information, as necessary. Then, when the iris information is used, authentication accuracy is increased. Therefore, according to the present example embodiment, while an advantage of convenience of face authentication is taken, master biometric information is easily registered.

Further, according to the present example embodiment, only when authentication using face information fails after a master iris image is registered, an authentication result using iris information is used. Therefore, authentication accuracy and convenience can be achieved with each other.

### Second Example Embodiment

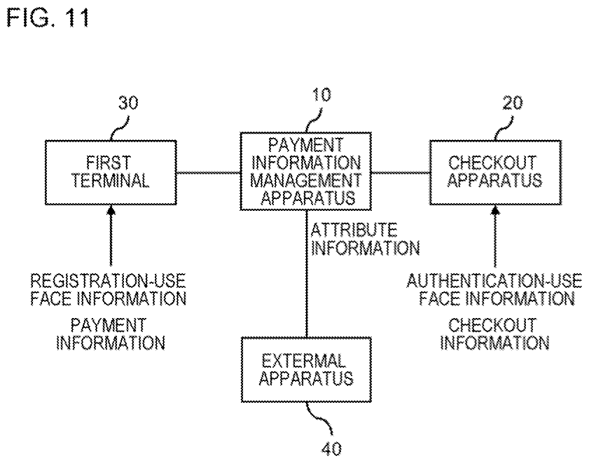

FIG. 11 is a diagram illustrating a usage environment of a payment information management apparatus 10 according to a second example embodiment. According to the present example embodiment, the payment information management apparatus 10 is similar to the payment information management apparatus 10 according to the first example embodiment, except that attribute information of a customer can be also acquired from an external apparatus 40.

The external apparatus 40 is, for example, an apparatus that manages payment information, and stores, in association with identification information (e.g., a number) of a credit card or an electronic money, attribute information of a user of the credit card or the electronic money. Then, a first registration unit 110 of the payment information management apparatus 10 acquires, from the external apparatus 40, attribute information relevant to payment information transmitted from a first terminal 30, and stores the acquired attribute information in a first storage unit 120.

According to the present example embodiment, a person may not necessarily input, in step S20 in FIG. 7, attribute information to the first terminal 30. Therefore, a load on a person decreases.

While with reference to the accompanying drawings, the example embodiments according to the present invention have been described, the example embodiments are illustrative of the present invention and various configurations other than the above-described configurations are employable.

Further, in a plurality of flowcharts used in the above-described description, a plurality of steps (pieces of processing) are described in order, but an execution order of steps to be executed according to each example embodiment is not limited to the described order. According to each example embodiment, an order of illustrated steps can be modified within an extent that there is no harm in context. Further, the above-described example embodiments can be combined within an extent that there is no conflict in content.

A part or whole of the above-described example embodiments may also be described as the following supplementary notes, but is not limited thereto.

1. A payment information management apparatus including:

a first registration unit that acquires, from a first terminal, payment information being information necessary for electronically making payment and master face information indicating a face of a person relevant to the payment information, and stores the payment information and the master face information in a first storage unit in association with each other;

an acquisition unit that acquires, from a checkout apparatus installed in a store, authentication-use face information being face information of a customer about to make a checkout by using the checkout apparatus;

an authentication unit that reads, when the master face information in which a score for the authentication-use face information is equal to or more than a reference value is present, the payment information relevant to the master face information, and causes the checkout apparatus to execute checkout processing using the payment information; and

a second registration unit that stores, in the first storage unit in association with the payment information of the customer, as master biometric information, biometric information being information generated by the checkout apparatus and including iris information and/or vein information of the customer, under a necessary condition in that the checkout apparatus executes the checkout processing using the payment information.

2. The payment information management apparatus according to supplementary note 1, wherein

the second registration unit further stores, in the first storage unit, the iris information and/or the vein information of the customer, under a necessary condition in that the first storage unit does not store the master biometric information in association with the payment information of the customer.

3. The payment information management apparatus according to supplementary note 1 or 2, wherein

the first registration unit further acquires terminal identification information necessary for communicating with a terminal of the person, and stores, in the first storage unit, the terminal identification information in association with the payment information, and

the second registration unit
reads, from the first storage unit, the terminal identification information relating to the payment information read by the authentication unit, transmits, to the terminal, information for confirming whether to be able to perform authentication processing using the biometric information by using the terminal identification information, and
stores, in the first storage unit, when acquiring information that the authentication processing is allowed from the terminal, information for setting the biometric information as the master biometric information.
   4. The payment information management apparatus according to supplementary note 3, wherein
the terminal is the first terminal.
   5. The payment information management apparatus according to supplementary note 3 or 4, wherein,

after the second registration unit causes the authentication processing using the biometric information to be executable,

the authentication unit executes, when the master face information in which a score for the authentication-use face information is equal to or more than a reference value is absent, the authentication processing using the biometric information of the customer, reads, when the authentication processing is successful, the payment information relevant to the iris information used for authentication, and causes the checkout apparatus to execute checkout processing using the payment information.

6. The payment information management apparatus according to any one of supplementary notes 1 to 5, wherein

the checkout apparatus performs at least a part of acquisition processing for the biometric information in parallel with at least a part of acquisition processing for the authentication-use face information, and

the acquisition unit acquires the biometric information together with the authentication-use face information.

7. The payment information management apparatus according to any one of supplementary notes 1 to 6, wherein

the first registration unit acquires attribute information of the person, and stores the attribute information in the first storage unit in association with the master face information.

8. The payment information management apparatus according to supplementary note 7, wherein

the attribute information is stored in an external apparatus in association with the payment information, and

the first registration unit reads, from the external apparatus, the attribute information relevant to the acquired payment information, and stores the read attribute information in the first storage unit.

9. A payment information management apparatus

being used together with a first storage unit that stores payment information being information necessary for electronically making payment, master face information indicating a face of a person relevant to the payment information, and master biometric information indicating biometric information including iris information and/or vein information of the person in association with one another, the apparatus including:

an acquisition unit that acquires, from a checkout apparatus installed in a store, authentication-use face information being face information of a customer about to make a checkout by using the checkout apparatus and authentication-use biometric information being the biometric information of the customer; and

an authentication unit that performs authentication processing by using the first storage unit, and the authentication-use biometric information and/or the authentication-use face information, wherein

the authentication unit
reads, when the master face information in which a score for the authentication-use face information is equal to or more than a reference value is present, the payment information relevant to the master face information, and causes the checkout apparatus to execute checkout processing using the payment information, and
determines, when the master face information in which a score for the authentication-use face information is equal to or more than a reference value is absent, the master biometric information in which a score for the authentication-use biometric information is equal to or more than a reference value, reads the payment information relevant to the master biometric information, and causes the checkout apparatus to execute checkout processing using the payment information.
   10. A checkout system including:

a checkout apparatus used in a store; and

a payment information management apparatus that manages information used in the checkout apparatus, wherein

the payment information management apparatus includes
a first registration unit that acquires, from a first terminal, payment information being information necessary for electronically making payment and master face information indicating a face of a person relevant to the payment information, and stores the payment information and the master face information in a first storage unit in association with each other,

the checkout apparatus includes
a face information generation unit that generates authentication-use face information being face information of a customer about to make a checkout by using the checkout apparatus,
a biometric information generation unit that generates biometric information including iris information and/or vein information of the customer, and
a transmission unit that transmits, to the payment information management apparatus, the authentication-use face information and the biometric information, and

the payment information management apparatus further includes
an acquisition unit that acquires, from the checkout apparatus, the authentication-use face information and the biometric information,
an authentication unit that reads, when the master face information in which a score for the authentication-use face information is equal to or more than a reference value is present, the payment information relevant to the master face information, and causes the checkout apparatus to execute checkout processing using the payment information, and
a second registration unit that stores, in the first storage unit in association with the payment information of the customer, as master biometric information, the biometric information acquired from the checkout apparatus, under a necessary condition in that the checkout apparatus executes the checkout processing using the payment information.
   11. A payment information management method including,

by a computer:
acquiring, from a first terminal, payment information being information necessary for electronically making payment and master face information indicating a face of a person relevant to the payment information and storing the payment information and the master face information in a first storage unit in association with each other;
acquiring, from a checkout apparatus installed in a store, authentication-use face information being face information of a customer about to make a checkout by using the checkout apparatus;
reading, when the master face information in which a score for the authentication-use face information is equal to or more than a reference value is present, the payment information relevant to the master face information, and causing the checkout apparatus to execute checkout processing using the payment information; and
storing, in the first storage unit in association with the payment information of the customer, as master biometric information, biometric information being information generated by the checkout apparatus and including iris information and/or vein information of the customer, under a necessary condition in that the checkout apparatus executes the checkout processing using the payment information.
   12. The payment information management method according to supplementary note 11, further including,

by the computer,

storing, in the first storage unit, the iris information and/or the vein information of the customer, under a necessary condition in that the first storage unit does not store the master biometric information in association with the payment information of the customer.

13. The payment information management method according to supplementary note 11 or 12, further including,

by the computer:
acquiring terminal identification information necessary for communicating with a terminal of the person, and storing, in the first storage unit, the terminal identification information in association with the payment information;
reading, from the first storage unit, the terminal identification information relating to the read payment information, and transmitting, to the terminal, information for confirming whether to be able to perform authentication processing using the biometric information by using the terminal identification information; and

storing, in the first storage unit, when acquiring information that the authentication processing is allowed from the terminal, information for setting the biometric information as the master biometric information.

14. The payment information management method according to supplementary note 13, wherein

the terminal is the first terminal.

15. The payment information management method according to supplementary note 13 or 14, further including,

by the computer,

after causing the authentication processing using the biometric information to be executable, executing, when the master face information in which a score for the authentication-use face information is equal to or more than a reference value is absent, the authentication processing using the biometric information of the customer, reading, when the authentication processing is successful, the payment information relevant to the iris information used for authentication, and causing the checkout apparatus to execute checkout processing using the payment information.

16. The payment information management method according to any one of supplementary notes 11 to 15, wherein

the checkout apparatus performs at least a part of acquisition processing for the biometric information in parallel with at least a part of acquisition processing for the authentication-use face information,

the payment information management method further including,

by the computer,

acquiring the biometric information together with the authentication-use face information.

17. The payment information management method according to any one of supplementary notes 11 to 16, further including,

by the computer,

acquiring attribute information of the person, and storing the attribute information in the first storage unit in association with the master face information.

18. The payment information management method according to supplementary note 17, wherein

the attribute information is stored in an external apparatus in association with the payment information,

the payment information management method further including,

by the computer,

reading, from the external apparatus, the attribute information relevant to the acquired payment information, and storing the read attribute information in the first storage unit.

19. A program causing a computer to include:

a first registration function of acquiring, from a first terminal, payment information being information necessary for electronically making payment and master face information indicating a face of a person relevant to the payment information, and storing the payment information and the master face information in a first storage unit in association with each other;

an acquisition function of acquiring, from a checkout apparatus installed in a store, authentication-use face information being face information of a customer about to make a checkout by using the checkout apparatus;

an authentication function of reading, when the master face information in which a score for the authentication-use face information is equal to or more than a reference value is present, the payment information relevant to the master face information, and causing the checkout apparatus to execute checkout processing using the payment information; and

a second registration function of storing, in the first storage unit in association with the payment information of the customer, as master biometric information, biometric information being information generated by the checkout apparatus and including iris information and/or vein information of the customer, under a necessary condition in that the checkout apparatus executes the checkout processing using the payment information.

20. The program according to supplementary note 19, wherein

the second registration function further stores, in the first storage unit, the iris information and/or the vein information of the customer, under a necessary condition in that the first storage unit does not store the master biometric information in association with the payment information of the customer.

21. The program according to supplementary note 19 or 20, wherein

the first registration function
further acquires terminal identification information necessary for communicating with a terminal of the person and stores, in the first storage unit, the terminal identification information in association with the payment information,
reads, from the first storage unit, the terminal identification information relating to the payment information read by the authentication function, transmits, to the terminal, information for confirming whether to be able to perform authentication processing using the biometric information by using the terminal identification information, and
stores, in the first storage unit, when acquiring information that the authentication processing is allowed from the terminal, information for setting the biometric information as the master biometric information.
   22. The program according to supplementary note 21, wherein

the terminal is the first terminal.

23. The program according to supplementary note 21 or 22, wherein,

after the second registration function causes the authentication processing using the biometric information to be executable,

the authentication function executes, when the master face information in which a score for the authentication-use face information is equal to or more than a reference value is absent, the authentication processing using the biometric information of the customer, reads, when the authentication processing is successful, the payment information relevant to the iris information used for authentication, and causes the checkout apparatus to execute checkout processing using the payment information.

24. The program according to any one of supplementary notes 19 to 23, wherein

the checkout apparatus performs at least a part of acquisition processing for the biometric information in parallel with at least a part of acquisition processing for the authentication-use face information, and

the acquisition function acquires the biometric information together with the authentication-use face information.

25. The program according to any one of supplementary notes 19 to 24, wherein

the first registration function acquires attribute information of the person, and stores the attribute information in the first storage unit in association with the master face information.

26. The program according to supplementary note 25, wherein

the attribute information is stored in an external apparatus in association with the payment information, and

the first registration function reads, from the external apparatus, the attribute information relevant to the acquired payment information, and stores the read attribute information in the first storage unit.

This application is based upon and claims the benefit of priority from Japanese patent application No. 2020-034919, filed on Mar. 2, 2020, the disclosure of which is incorporated herein in its entirety by reference.

### REFERENCE SIGNS LIST

10 Payment information management apparatus
20 Checkout apparatus
30 First terminal
40 External apparatus
110 First registration unit
120 First storage unit
130 Acquisition unit
140 Authentication unit
150 Second registration unit
210 Face information generation unit
220 Biometric information generation unit
230 Checkout processing unit
310 Face information generation unit
320 Registration unit
330 Approval unit

What is claimed is:

1. A payment information management apparatus comprising:
at least one memory configured to store instructions; and
at least one processor configured to execute the instructions to preform operations comprising:
acquiring, from a first terminal, payment information being information necessary for electronically making payment and master face information indicating a face of a person relevant to the payment information;
storing the payment information and the master face information in a first storage in association with each other;
acquiring, from a checkout apparatus installed in a store, authentication-use face information being face information of a customer about to make a checkout by using the checkout apparatus;
reading, when the master face information in which a score for the authentication-use face information is equal to or more than a reference value is present, the payment information relevant to the master face information;
causing the checkout apparatus to execute checkout processing using the payment information; and
storing, in the first storage in association with the payment information of the customer, as master biometric information, biometric information being information generated by the checkout apparatus and including iris information and/or vein information of the customer, under a necessary condition in that the checkout apparatus executes the checkout processing using the payment information.

2. The payment information management apparatus according to claim 1, wherein the operations further comprise
storing, in the first storage, the iris information and/or the vein information of the customer, under a necessary condition in that the first storage does not store the master biometric information in association with the payment information of the customer.

3. The payment information management apparatus according to claim 1, wherein the operations further comprise:
acquiring terminal identification information necessary for communicating with a terminal of the person;
storing, in the first storage, the terminal identification information in association with the payment information;
reading, from the first storage, the terminal identification information relating to the payment information;
transmitting, to the terminal, information for confirming whether to be able to perform authentication processing using the biometric information by using the terminal identification information; and
storing, in the first storage, when acquiring information that the authentication processing is allowed from the terminal, information for setting the biometric information as the master biometric information.

4. The payment information management apparatus according to claim 3, wherein
the terminal is the first terminal.

5. The payment information management apparatus according to claim 3, wherein the operations further comprise,
after causing the authentication processing using the biometric information to be executable,
executing, when the master face information in which a score for the authentication-use face information is equal to or more than a reference value is absent, the authentication processing using the biometric information of the customer;
reading, when the authentication processing is successful, the payment information relevant to the iris information used for authentication; and
causing the checkout apparatus to execute checkout processing using the payment information.

6. The payment information management apparatus according to claim 1, wherein
the checkout apparatus performs at least a part of acquisition processing for the biometric information in parallel with at least a part of acquisition processing for the authentication-use face information, and
the operations further comprise acquiring the biometric information together with the authentication-use face information.

7. The payment information management apparatus according to claim 1, wherein the operations further comprise
acquiring attribute information of the person; and
storing the attribute information in the first storage in association with the master face information.

8. The payment information management apparatus according to claim 7, wherein
the attribute information is stored in an external apparatus in association with the payment information, and
the operations further comprise reading, from the external apparatus, the attribute information relevant to the acquired payment information, and storing the read attribute information in the first storage.

9. (canceled)

10. A checkout system comprising:
a checkout apparatus used in a store; and
a payment information management apparatus that manages information used in the checkout apparatus, wherein
the payment information management apparatus comprises
at least one memory configured to store instructions; and
at least one processor configured to execute the instructions to preform first operations comprising:
acquiring, from a first terminal, payment information being information necessary for electronically making payment and master face information indicating a face of a person relevant to the payment information; and
storing the payment information and the master face information in a first storage in association with each other,
the checkout apparatus comprises:
at least one memory configured to store instructions; and
at least one processor configured to execute the instructions to preform second operations comprising:
generating authentication-use face information being face information of a customer about to make a checkout by using the checkout apparatus;
generating biometric information including iris information and/or vein information of the customer; and
transmitting, to the payment information management apparatus, the authentication-use face information and the biometric information, and
the first operations further comprises:
acquiring, from the checkout apparatus, the authentication-use face information and the biometric information;
reading, when the master face information in which a score for the authentication-use face information is equal to or more than a reference value is present, the payment information relevant to the master face information;
causing the checkout apparatus to execute checkout processing using the payment information; and
storing, in the first storage unit in association with the payment information of the customer, as master biometric information, the biometric information acquired from the checkout apparatus, under a necessary condition in that the checkout apparatus executes the checkout processing using the payment information.

11. A payment information management method comprising,
by a computer:
acquiring, from a first terminal, payment information being information necessary for electronically making payment and master face information indicating a face of a person relevant to the payment information, and storing the payment information and the master face information in a first storage unit in association with each other;
acquiring, from a checkout apparatus installed in a store, authentication-use face information being face information of a customer about to make a checkout by using the checkout apparatus;
reading, when the master face information in which a score for the authentication-use face information is equal to or more than a reference value is present, the payment information relevant to the master face information, and causing the checkout apparatus to execute checkout processing using the payment information; and
storing, in the first storage unit in association with the payment information of the customer, as master biometric information, biometric information being information generated by the checkout apparatus and including iris information and/or vein information of the customer, under a necessary condition in that the checkout apparatus executes the checkout processing using the payment information.

12-26. (canceled)
